(19) United States
(12) Patent Application Publication
(10) Pub. No.: US 2026/0240172 A1
(43) Pub. Date: Aug. 20, 2026

(54) LIPOPEPTIDE COMPOSITION FOR PROMOTING ROOT NODULE GROWTH

(71) Applicant: BEIJING EVOLYZER CO., LTD., Beijing (CN)

(72) Inventors: Miaomiao WANG, Beijing (CN); Bo CHEN, Beijing (CN)

(73) Assignee: BEIJING EVOLYZER CO., LTD., Beijing (CN)

(21) Appl. No.: 19/164,853

(22) PCT Filed: Feb. 27, 2024
(86) PCT No.: PCT/CN2024/078734

§ 371 Date: Sep. 12, 2025

(30) Foreign Application Priority Data
Mar. 15, 2023 (CN) 202310266693.8

(51) Int. Cl. A01N 63/50; A01N 63/22; A01P 21/00; C07K 7/56

(52) U.S. Cl. CPC A01N 63/50; A01N 63/22; A01P 21/00; C07K 7/56

(57) ABSTRACT

The present application provides a composition for agriculture, in particular for promoting root nodule growth, wherein the composition comprises 0.001-100 g/L of lipopeptide. The present application also provides Use of the composition for promoting root nodule growth. Specifically, compared with a control group without applying the composition, the root nodule quantity and the root nodule weight of each plant applying the composition are significantly increased.

### TECHNICAL FIELD

The present disclosure relates to the fields of biotechnology and biochemical engineering, specifically to lipopeptide compositions and use thereof for promoting root nodule growth, and specifically to lipopeptide compositions and use thereof for promoting root nodule growth.

### BACKGROUND

Lipopeptides are a class of amphiphilic substances formed by the connection of hydrophilic cyclic oligopeptides with hydrophobic fatty acid chains through lactone bonds. They are mainly synthesized by microorganisms such as Bacillus, Pseudomonas, and Streptomyces, and have biological activities such as antibacterial and antiviral activities.

According to the different types, numbers, and ring-forming groups of amino acids in the peptide ring, lipopeptides can be classified into surfactin, fengycin, iturin and the like. Typically, surfactin is composed of β-hydroxy fatty acids having 12-17 carbon atoms in length together with 7 α-amino acids. Fengycin is composed of a β-hydroxy fatty acid having 14-17 carbon atoms in length together with 10 amino acids. Iturin is composed of β-amino fatty acid having 14-17 carbon atoms in length together with a peptide chain of heptapeptides.

Symbiotic nitrogen fixation is a unique phenomenon in nature where plants interact with microorganisms, having significant importance for agricultural production. Currently, in the cultivation and management of leguminous plants, cannabis plants, or other plants that fix nitrogen through symbiosis with microorganisms, the primary approach is to improve the plant's growth environment to increase the number of root nodules, thereby enhancing their nitrogen-fixing capacity. The approach is primarily categorized into chemical, agricultural, and biological measures: chemical measures involve applying chemical nitrogen fertilizers during cultivation; agricultural measures include increasing the application of organic fertilizers, increasing the frequency of intertillage, appropriate irrigation, reasonable supplementary application of micronutrient fertilizers, and adjusting pH of soil; biological measures generally involve seed dressing cultivation by using Rhizobium inoculant, developing engineered nitrogen-fixing microorganisms, and making them assist plants in nitrogen fixation to promote plant growth.

Among the above measures, long-term use of chemical nitrogen fertilizers will not only affect nitrogen fertilizer utilization rates, but excessive use can also damage the environment and generate large amounts of carbon emissions. Appropriate agricultural measures can slightly increase the number of root nodules, but they are easily affected by factors such as uncontrollable climate and imprecise management timing, which can lead to improper timing of root nodule formation and weak nitrogen fixation capacity in plants.

The method of seed dressing with Rhizobium inoculant (mixing and agigating rhizobia fermentation liquid and substrate) can enhance crop yields when applied to the soils that have never been used for legume cultivation. However, during the seed dressing process, sterilizing substrates such as peat is complex, prone to contamination by foreign bacteria, and results in unstable rhizobia content, making it difficult to ensure the the seed dressing quality of the inoculant. This limits its widespread application in agriculture. To address this issue, people in many regions use insecticides and bactericides/fungicides as seed coating agents for seed treatment before planting legume seeds. However, most pesticides are toxic to rhizobia, directly causing functional conflict between Rhizobium inoculant and insecticide/bactericide/fungicide seed coatings. This is also the current bottleneck problem which hinders the widespread application of Rhizobium inoculants.

In order to address the various issues currently involved in promoting the growth of plants and their nitrogen-fixing symbionts, this field urgently requires a new type of composition that can promote the formation and weight of plant nodules, is environmentally friendly, minimizes chemical and biological pollution, and is safe for long-term use.

### REFERENCES

1. CN201580077068.2;
2. IN201717025497;
3. ARP20150103136;
4. CN201310561701.8.

### SUMMARY

Specifically, the present application has solved the above-mentioned technical problems present in the prior art by the following technical solutions.

1. A composition for promoting root nodule growth, comprising:
(i) 0.001-100 g/L (concentration) of lipopeptide; and
(ii) an adjuvant.

2. The composition according to item 1, wherein the lipopeptide is cyclic or linear lipopeptide, preferably, the cyclic lipopeptide has the following formulas (I) to (III):

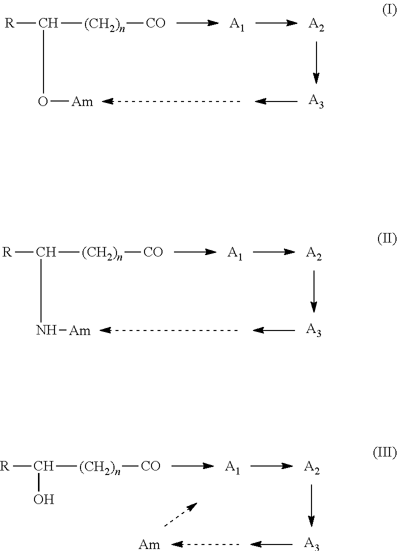

wherein R represents the carbon chain of the fatty acid chain; A₁, A₂, A₃ . . . Aₘ represent the 1^{st}, 2^{nd}, 3^{rd} . . . m^{th} amino acids on the peptide chain, respectively; the carboxyl group of the fatty acid is connected to the N-terminus of A₁, and the C-terminal carboxyl group of Aₘ is connected to the hydroxyl group or amino group of the fatty acid or other amino acids on the peptide chain to form a cyclic structure; the linear lipopeptide is a linear product obtained by opening the ring of cyclic lipopeptide.

3. The composition according to item 1 or 2, wherein the lipopeptide is one or more selected from a group consisting of surfactin family, iturin family and fengycin family, preferably the lipopeptide is one or more selected from a group consisting of surfactin, iturin, fengycin, lichenysin, bamylocin, pumilacidin, bacillomycin, mycosubtilin, plipastatin, fusaricidin, kurstakin, paenibacterin, polymyxin, octapeptin, bacaucin and polypeptin.

4. The composition according to any one of items 1 to 3, wherein the lipopeptide is produced by bacteria, actinomycetes, fungi, or cyanobacteria; preferably the bacteria are wild-type bacteria or genetically engineered bacteria, and more preferably the bacteria are selected from the group consisting of Bacillus, Pseudomonas, Streptomyces, Arthrobacter and Burkholderia; more preferably the actinomycetes are selected from the group consisting of Streptomyces, Amycolatopsis and Microbacterium; and more preferably the fungi are selected from the group consisting of Aspergillus, Microascus, Stachylidium, Myrothecium, Sarocladium, Ophiosphaerella and Clavariopsis; and more preferably the cyanobacteria are selected from the group consisting of Okeania, Cladonia and Moorea; more preferably, the lipopeptides are obtained by cultivating Bacillus or Pseudomonas; and more preferably, the lipopeptides are the ones obtained by cultivating Bacillus and regulating the expression of genes related to lipopeptide synthesis; and more preferably, the genes related to lipopeptide synthesis are selected from the group consisting of: lipopeptide synthesis genes, transmembrane transport protein gene ycxA, biotin carboxylase gene yngH, spore synthesis genes spoIVA/B/C/F, spoVA/B/D/E, and leucine synthesis pathway genes leuABCD/ilvK.

5. The composition according to item 4, wherein the lipopeptide synthesis genes are selected from the group consisting of: surfactin synthesis gene srfA, fengycin synthesis gene fen, lichenysin synthesis gene lic, bacillomycin synthesis gene bam, mycosubtilin myc, iturin synthesis gene itu, plipastatin synthesis gene pps, fusaricidinfus, kurstakin krs, polymyxin pmx and octapeptin oct.

6. The composition according to any one of items 1 to 5, further comprising 0.01-99.99 wt % of one or more components selected from the group consisting of: polysaccharides, oligosaccharides, dextrins, amino acids, oligopeptides, proteins, lipids, fatty acids or the derivatives thereof, inorganic salts, dispersants, wetting agents, polymers, and warning colorants.

7. The composition according to any one of items 1 to 6, wherein the composition is a culture solution obtained by cultivating bacteria capable of producing lipopeptides.

8. The composition according to any one of 1 to 7, the root nodule is produced by bacteria, actinomycetes, fungi, or cyanobacteria that are symbiotic with plants, wherein preferably the bacteria are selected from α-, β-, or γ-Proteobacteria, and more preferably are selected from the group consisting of: Rhizobium, Sinorhizobium, Ensifer, Shinella, Neorhizobium, Pararhizobium, Mesorhizobium, Bradyrhizobium, Phyllobacterium, Methylobacterium, Microvirga, Ocrhobactrum, Azorhizobium, Devosia, Burkholderia, Cupriavidu and Pseudomonas; preferably the actinomycetes are Frankia; preferably the fungi are Ascomycetes and preferably Basidiomycetes; the cyanobacteria are Nostoc and Anabaena.

9. The composition according to item 8, wherein the the root nodule is produced by bacteria that are symbiotic with plants, and the plants are leguminous or cannabis plants, preferably selected from the group consisting of: soybeans, broad beans, peas, mung beans, adzuki beans, cowpeas, kidney beans, lentils, winged beans, black beans, chickpeas, peanuts, purple vetch, alfalfa, Chinese milk vetch, acacia, Dalbergia hupeana Hance, Chinese honey locust, Erythrophleum fordii Oliv., Sophora japonica, Indigofera pseudotinctoria, Indigofera tinctoria L, sappan wood, arabic tree gum, Astragalus membranaceus, copal gum tree, Crotalaria juncea L., kudzu vine, Trema tomentosa, and Parasponia.

10. The composition according to item 8, the the root nodule is produced by Frankia, cyanobacteria or fungi that are symbiotic with plants, and the plants are selected from the group consisting of: Betulaceae, Casuarinaceae, Myricaceae, Rhamnaceae, Elaeagnaceae, Datiscaceae, Coriariaceae, Rosaceae, Bryophytes, ferns and cycades, Nostoc, Coccidium, Trentepohlia, Scytonema and Scytonema.

11. A method for preparing the composition according to any one of items 1 to 10, comprising:
cultivating the bacteria capable of producing lipopeptides under conditions suitable for lipopeptide production to obtain a culture solution containing 0.001-100 g/L of lipopeptides, and using the culture solution as the composition; or
formulating purified or partially purified lipopeptides into solutions or solid mixtures containing 0.001-100 g/L of lipopeptides, and using the solutions or solid mixtures as the composition.

12. The method according to item 11, further comprising: to the ulture medium, solutions or solid mixtures, adding 0.01-99.99 wt % of one or more components selected from the group consisting of: polysaccharides, oligosaccharides, dextrins, amino acids, oligopeptides, proteins, lipoids, fatty acids or the derivatives thereof, inorganic salts, dispersants, wetting agents, polymers, and warning colorants; and using the resulting product as the composition.

13. The method according to item 11 or 12, further comprising:
concentrating and/or drying and/or partially purifying the culture solution, solution, solid mixture, or product, and using the concentrated and/or dried and/or partially purified product as the composition.

14. A method for promoting root nodule growth by using the composition according to any one of items 1 to 10, comprising:
(1) directly applying the composition to the culture of the plants that are symbiotic with bacteria, actinomycetes, fungi, or cyanobacteria which produce the root nodule;
(2) cultivating and harvesting the plants;
(3) optionally measuring the parameters of the root nodule, and comparing them with those of the control group to which no composition is applied, preferably, the parameters are selected from the group consisting of: the number of root nodules and root nodule weight of each plant.

15. The method according to item 14, wherein the promotion of root nodule growth is characterized by one or more features selected from the group consisting of:
(1) the number of root nodules per plant of the plants cultivated by applying the composition is at least 1.1 times, at least 1.2 times, at least 1.3 times, at least 1.4 times, at least 1.5 times, at least 1.6 times, at least 1.7 times, at least 1.8 times, at least 1.9 times, at least 2 times, preferably at least 3 times, more preferably at least 4 times, more preferably at least 5 times, more preferably at least 6 times, more preferably at least 7 times that of the control group; and/or
(2) the root nodule weight per plant of the plants cultivated by applying the composition is at least 1.1 times, at least 1.2 times, at least 1.3 times, at least 1.4 times, at least 1.5 times, at least 1.6 times, at least 1.7 times, at least 1.8 times, at least 1.9 times, at least 2 times, preferably at least 3 times, more preferably at least 4 times, more preferably at least 5 times, more preferably at least 6 times, more preferably at least 7 times that of the control group.

16. The method according to item 14 or 15, which meanwhile promotes the growth of the plants that are symbiotic with bacteria, actinomycetes, fungi, or cyanobacteria which produce the root nodule, preferably the promotion of the growth of the plants is characterized by the plant height of the aboveground part, the fresh weight of the aboveground part, the fresh weight of the underground part, and the number or weight of plant products, more preferably is characterized by one or more features selected from the group consisting of:
(1) the plant height of the aboveground part of the plants cultivated by applying the composition increases 10% or more, preferably 15% or more, more preferably 20% or more, more preferably 25% or more, more preferably 30% or more, more preferably 35% or more, more preferably 36% or more, as compared to the control group;
(2) the fresh weight of the aboveground part of the plants cultivated by applying the composition increases 15% or more, preferably 20% or more, more preferably 25% or more, more preferably 30% or more, more preferably 35% or more, more preferably 40% or more, more preferably 45% or more, more preferably 47% or more, as compared to the control group;
(3) the fresh weight of the underground part of the plants cultivated by applying the composition increases 5% or more, preferably 8% or more, more preferably 10% or more, more preferably 12% or more, as compared to the control group; and/or
(4) the number or weight of plant products cultivated by applying the composition is at least 1.1 times, preferably at least 1.3 times, more preferably at least 1.6 times, more preferably at least 1.9 times, more preferably at least 2.3 times, more preferably at least 3.0 times that of the control group, preferably the plant products are selected from a group consisting of: fruit pods, pegs and seeds of the plant.

17. The method according to any one of items 14 to 16, wherein the application is selected from a group consisting of: seed coating, seed soaking treatment, bulb treatment, root irrigation treatment, drip irrigation treatment, and foliar spraying application.

18. Use of the composition according to any one of items 1 to 10 for promoting root nodule growth.

19. Use of the composition according to any one of items 1 to 10 in agricultural fertilizers, agricultural adjuvants or pesticides.

20. Use of the composition according to any one of items 1 to 10 for promoting root nodule growth and meanwhile promoting the growth of the plants that are symbiotic with bacteria, actinomycetes, fungi, or cyanobacteria which produce the root nodule.

21. Use of lipopeptide for promoting root nodule growth.

22. Use of lipopeptide in agricultural fertilizers, agricultural adjuvants or pesticides.

23. Use of lipopeptide for promoting root nodule growth and meanwhile promoting the growth of the plants that are symbiotic with bacteria, actinomycetes, fungi, or cyanobacteria which produce the root nodule.

24. The use according to any one of items 21 to 23, wherein the lipopeptide is one or more selected from a group consisting of: surfactin, iturin, fengycin, lichenysin, bamylocin, pumilacidin, bacillomycin, mycosubtilin, plipastatin, fusaricidin, kurstakin, paenibacterin, polymyxin, octapeptin, bacaucin and polypeptin.

25. The use according to item 24, wherein the lipopeptide is one or more selected from a group consisting of: surfactin, iturin and fengycin.

26. The use according to any one of items 21 to 25, wherein the lipopeptide is produced by bacteria, actinomycetes, fungi, or cyanobacteria; preferably the the bacteria are wild-type bacteria or genetically engineered bacteria, and more preferably the bacteria are selected from the group consisting of Bacillus, Pseudomonas, Streptomyces, Arthrobacter and Burkholderia; more preferably the actinomycetes are selected from the group consisting of Streptomyces, Amycolatopsis and Microbacterium; and more preferably the fungi are selected from the group consisting of Aspergillus, Microascus, Stachylidium, Myrothecium, Sarocladium, Ophiosphaerella and Clavariopsis; and more preferably the cyanobacteria are selected from the group consisting of Okeania, Cladonia and Moorea; more preferably, the lipopeptides are obtained by cultivating Bacillus or Pseudomonas; and more preferably, the lipopeptides are the ones obtained by cultivating Bacillus and regulating the expression of genes related to lipopeptide synthesis; and more preferably, the genes related to lipopeptide synthesis are selected from the group consisting of: lipopeptide synthesis genes, transmembrane transport protein gene ycxA, biotin carboxylase gene yngH, spore synthesis genes spoIVA/B/C/F, spoVA/B/D/E, and leucine synthesis pathway genes leuABCD/ilvK.

27. The use according to item 26, wherein the lipopeptide synthesis genes are selected from the group consisting of: surfactin synthesis gene srfA, fengycin synthesis gene fen, lichenysin synthesis gene lic, bacillomycin synthesis gene bam, mycosubtilin myc, iturin synthesis gene itu, plipastatin synthesis gene pps, fusaricidinfus, kurstakin krs, polymyxin pmx and octapeptin oct.

28. The use according to any one of items 21 to 27, the root nodule is produced by bacteria, actinomycetes, fungi, or cyanobacteria that are symbiotic with plants, wherein preferably the bacteria are selected from α-, β-, or γ-Proteobacteria, and more preferably are selected from the group consisting of: Rhizobium, Sinorhizobium, Ensifer, Shinella, Neorhizobium, Pararhizobium, Mesorhizobium, Bradyrhizobium, Phyllobacterium, Methylobacterium, Microvirga, Ocrhobactrum, Azorhizobium, Devosia, Burkholderia, Cupriavidu and Pseudomonas; preferably the actinomycetes are Frankia; preferably the fungi are Ascomycetes and preferably Basidiomycetes; the cyanobacteria are Nostoc and Anabaena.

29. The use according to item 28, wherein the the root nodule is produced by bacteria that are symbiotic with plants, the plants are leguminous or cannabis plants, preferably selected from the group consisting of: soybeans, broad beans, peas, mung beans, adzuki beans, cowpeas, kidney beans, lentils, winged beans, black beans, chickpeas, peanuts, purple vetch, alfalfa, Chinese milk vetch, acacia, Dalbergia hupeana Hance, Chinese honey locust, Erythrophleum fordii Oliv., Sophora japonica, Indigofera pseudotinctoria, Indigofera tinctoria L, sappan wood, arabic tree gum, Astragalus membranaceus, copal gum tree, Crotalaria juncea L., kudzu vine, Trema tomentosa, and Parasponia.

30. The use according to item 28, wherein the the root nodule is produced by Frankia, cyanobacteria or fungi that are symbiotic with plants, the plants are selected from the group consisting of: Betulaceae, Casuarinaceae, Myricaceae, Rhamnaceae, Elaeagnaceae, Datiscaceae, Coriariaceae, Rosaceae, Bryophytes, ferns and cycades, Nostoc, Coccidium, Trentepohlia, Scytonema and Scytonema.

The present embodiments can be understood more clearly and fully by further describing the present application with reference to the figures and specific embodiments.

### BRIEF DESCRIPTION OF THE DRAWINGS

FIG. 1 shows the growth picture of the aboveground part of soybean grown in potted plants;

FIG. 2 shows the growth picture of the underground part of soybean grown in potted plants;

FIG. 3 shows the growth picture of the root system of soybean grown in large field;

FIG. 4 shows the growth picture of the aboveground part of mung bean grown in large field;

FIG. 5 shows the picking picture of root nodule of mung bean grown in large field;

FIG. 6 shows the control picture of the yield of red bean; and

FIG. 7 shows the growth picture of the plant of peanut grown in large field.

### DETAILED DESCRIPTION

### 1. Definition

As used herein, the terms “lipopeptide”, “peptidolipid”, also referred to as acylpeptide, often refer to a peptide formed by the connection of fatty acids with peptide chains through ester bonds or amide bonds, and can be classified into cyclic lipopeptide and linear lipopeptide. Cyclic lipopeptides refer to lipopeptides with a cyclic structure, in which the carboxyl group of a fatty acid is connected to the N-terminus of an amino acid in the peptide chain, while the C-terminal carboxyl group of an amino acid in the peptide chain is connected to a fatty acid or to the hydroxyl or amino group of other amino acid in the peptide chain to form a cyclic structure. The ring-forming forms of cyclic lipopeptides include: ring formation of hydroxyl of fatty acid, ring formation of amino of fatty acid, and ring attachment or ring deattachment of fatty acid, which are represented by the following formulas, respectively:

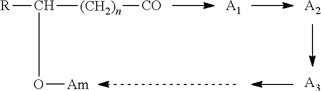

(I) ring formation of hydroxyl of fatty acid

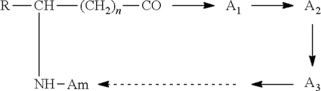

(II) ring formation of amino of fatty acid

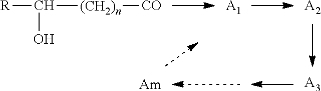

(III) ring attachment of fatty acid (Aₘ forms a ring with A₁) or ring deattachment of fatty acid (Aₘ forms a ring with amino acid other than A₁);
the linear lipopeptide is a linear product obtained by opening the ring of cyclic lipopeptide.

The lipopeptide is produced by bacteria, actinomycetes, fungi, or cyanobacteria, preferably by bacteria, more preferably by Bacillus, Pseudomonas, Streptomyces or Arthrobacter and the like (see Janek et al., 2010). Three major known lipopeptide families are the surfactin family, the iturin family, and the fengycin family. The lipopeptide can be one or more selected from the group consisting of: surfactin, iturin, fengycin, lichenysin, bamylocin, pumilacidin, bacillomycin, mycosubtilin, plipastatin, fusaricidin, kurstakin, paenibacterin, polymyxin, octapeptin, bacaucin and polypeptin. The main differences between them lie in the length of the fatty acid side chains, the isomerization patterns, and the types, numbers, and connection sequences of amino acids in the peptide rings.

The surfactin family comprises over 20 distinct molecules, which are cyclic peptides formed from C12-16 β-hydroxy fatty acids with a 7-amino acid condensed polypeptide via lactone bonds (Bonmatin et al., 2003). The heptapeptide segment exhibits a chiral sequence of LLDLLDL, and the chirality of D-Leu at positions 3 and 6 and L-amino acid at position 4 is strictly conservative, playing a key role in peptide chain closure (Peypoux et al., 1999), while substitutions of amino acid such as Leu, Val, Ile, Ala and the like can be observed at positions 2, 4, and 7 (Bonmatin et al., 1995; Peypoux et al., 1991; Peypoux et al., 1994). The molecular structure of surfactin is shown in the following formula (IV):

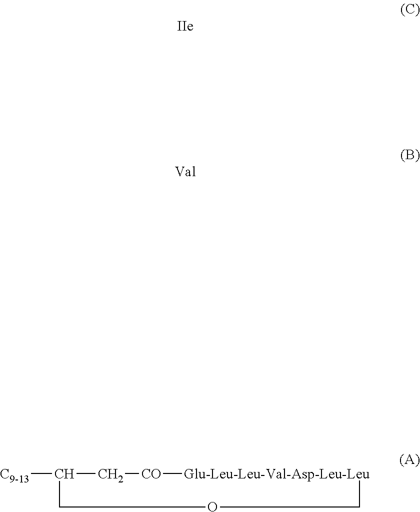

(IV) amino acid sequence and connection pattern of surfactin

The iturin family so far comprises 6 main members: iturin A, C; bacillomycin D, F, L; and mycosubtilin (Bonmatin et al., 2003). All of these are cyclic peptides formed from a C14-17 β-amino fatty acid with a 7-amino acid condensed polypeptide via a lactam bond. All members of the iturin family strictly conform to the LDDLLDL chiral conformation and contain a common amino acid sequence: β-amino fatty acid-L-Asx-D-Tyr-D-Asn (Peypoux et al., 1978). The molecular structure of iturin is shown in the following formula (V):

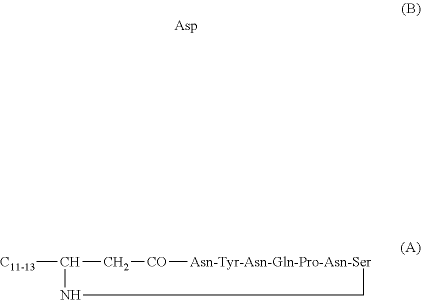

(V) amino acid sequence and connection pattern of iturin

The fengycin (also known as fengycin) family is the third largest lipopeptide family after the surfactin family and the iturin family. Members of the fengycin family are formed by the connection of C14-17 β-hydroxy fatty acids with the first position Glu of a decapeptide cyclic peptide via ester bond, with the lactone bond formed between the hydroxyl group of the 3^{rd} position Tyr and the carbon terminal of the 10^{th} position Ile (Nishikiori et al., 1986). The molecular structure of fengycin is shown in the following equation (VI):

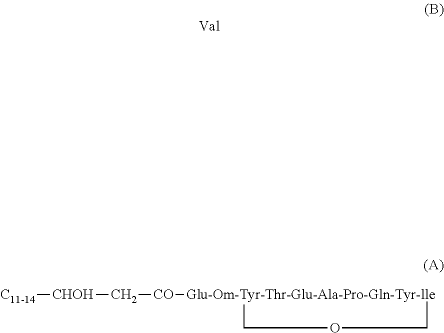

(VI) amino acid sequence and connection pattern of fengycin

As used herein, the terms “gene,” “enhanced expression,” “promoter,” “recombinant (bacteria),” “culture solution,” etc. are all conventional terms used in the field of molecular biology and have the same meanings as understood by those skilled in the art.

As used herein, the term “root nodule” refers to the tubercles grown on root system of a plant, which are produced by bacteria, actinomycetes, fungi, or cyanobacteria that are symbiotic with plants. The bacteria are the ones which are symbiotic with plants to form root nodule and are capable of fixing nitrogen, are preferably selected from α-, β-, or γ-Proteobacteria, and more preferably are selected from the group consisting of: Rhizobium, Sinorhizobium, Ensifer, Shinella, Neorhizobium, Pararhizobium, Mesorhizobium, Bradyrhizobium, Phyllobacterium, Methylobacterium, Microvirga, Ocrhobactrum, Azorhizobium, Devosia, Burkholderia, Cupriavidu and Pseudomonas. The bacteria settle in the roots of plants, stimulate certain cells in the root cortex and pericycle, causing these cells to grow vigorously and resulting in local enlargement of the root to form root nodules. Rhizobia fix free nitrogen in the atmosphere and provide nitrogenous nutrient to plants, while the plants provide nutrients and energy to root nodule. The two maintain a balanced state in an antagonistic parasitic relationship, exhibiting a symbiotic relationship. The nitrogen fixation role of root nodules in the growth process of leguminous plants cannot be replaced by any other technology or measures. Preferably The actinomycetes are Frankia, which are widely distributed in the root nodules of non-leguminous plants and form a symbiotic relationship with them. Frankia can include the following four groups: (1) Frankia (F. alni, F. casuarinae, F. canadensis) that are symbiotic with Betulaceae (Alnus Mill.), Casuarinaceae (Allocasuarina and Casuarina Adans), and Myricaceae; (2) Frankia (Candidatus Frankia datiscae, Candidatus Frankia californiensis) that are symbiotic with Coriariaceae, Datiscaceae, Rosaceae (Dryadoideae), Rhamnaceae (Rhamnus L); (3) Frankia (F. elaeagni, F. irregularis) that are symbiotic with Elaeagnaceae, Rhamnaceae (waxy fruit family), Myricaceae, Casuarinaceae (Gynmmostoma); and (4) other Frankia strain (F. inefficax, F. asymbiotica, F. saprophytica). The fungi are Ascomnycetes and Basidionycetes, and are capable of forming lichen through symbiosis with algae. The cyanobacteria, also known as blue-green algae, are a group of large unicellular prokaryotes with a long evolutionary history, which are Gram-negative, flagellum-free, contain chlorophyll a, but do not have chloroplasts (unlike eukaryotic algae), and are capable of performing aerobic photosynthesis.

As used herein, the term “plants which are symbiotic with Rhizobia” includes leguminous plants (Leguminosae), which are a family of dicotyledonous plants belonging to the order Rosales. They can be trees, shrubs, subshrubs, or herbaceous plants, either erect or climbing, and are often accompanied by nitrogen-fixing rhizobia. Leguminous plants are important economic crops, serving as a significant source of starch, protein, oil, and vegetables in the human diet. Leguminous plants include, but are not limited to: soybeans, broad beans, peas, mung beans, adzuki beans, cowpeas, kidney beans, lentils, winged beans, black beans, chickpeas, peanuts, purple vetch, alfalfa, Chinese milk vetch, acacia, Dalbergia hupeana Hance, Chinese honey locust, Erythrophleum fordii Oliv., Sophora japonica, Indigofera pseudotinctoria, Indigofera tinctoria L, sappan wood, arabic tree gum, Astragalus membranaceus, copal gum tree, Crotalaria juncea L., and kudzu vine.

As used herein, the term “plants which are symbiotic with Rhizobia” includes plants of the Cannabaceae family, which is a family of dicotyledonous plants belonging to the Rosales order. Plants of the Cannabaceae family include, but are not limited to the following genus: Gironniera Gaudich., Aphananthe Planch., cannabis, Celtis adolfi-friderici, humulus scandens, celtis, Pteroceltis Maxim., Lozanella permollis, Parasponia and trema.

As used herein, the term “plants which are symbiotic with actinomycetes” includes the plants of the following family: Betulaceae, Casuarinaceae, Myricaceae, Rhamnaceae, Elaeagnaceae, Datiscaceae, Coriariaceae and Rosaceae.

As used herein, the term “plants which are symbiotic with fungi” includes the algae of the following genus: Nostoc, Coccidium, Trentepohlia, Scytonema and Eucladia, and the like.

As used herein, the term “plants which are symbiotic with cyanobacteria” includes the plants such as Bryophytes, Salviniaceae or Cycadaceae.

As used herein, the term “adjuvant” includes but are not limited to: dispersants, wetting agents, binders, warning colorants, film-forming agents, fillers and the like in solid formulations; solvents, cosolvents, dispersants, wetting agents, emulsifiers, film-forming agents, preservatives, antioxidants, flavoring agents, aromatic agents, warning colorants in liquid mixtures.

### 2. Lipopeptide of the Present Application

The lipopeptide of the present application is cyclic lipopeptide or linear lipopeptide. In one embodiment, the lipopeptide of the present application is a cyclic lipopeptide having one of the following formulas (I)-(III):

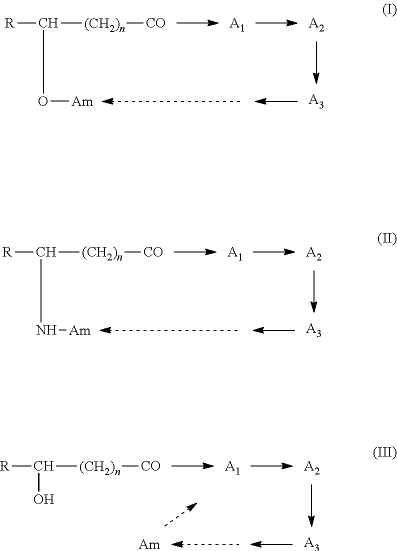

wherein R represents the carbon chain of the fatty acid chain; A₁, A₂, A₃ . . . Aₘ represent the 1^{st}, 2^{nd}, 3^{rd} . . . m^{th} amino acids on the peptide chain, respectively; the carboxyl group of the fatty acid is connected to the N-terminus of A₁, and the C-terminal carboxyl group of Aₘ is connected to the hydroxyl group or amino group of the fatty acid or other amino acids on the peptide chain to form a cyclic structure. In one embodiment, m is an integer of 6-20. In one embodiment, the lipopeptide of the present application is selected from a group consisting of: surfactin family, iturin family and fengycin family. In one embodiment, the lipopeptide of the present application is one or more selected from a group consisting of: surfactin, iturin, fengycin, lichenysin, bamylocin, pumilacidin, bacillomycin, mycosubtilin, plipastatin, fusaricidin, kurstakin, paenibacterin, polymyxin, octapeptin, bacaucin and polypeptin. one or more selected from a group consisting of surfactin, iturin, fengycin, lichenysin, bamylocin, pumilacidin, bacillomycin, mycosubtilin, plipastatin, fusaricidin, kurstakin, paenibacterin, polymyxin, octapeptin, bacaucin and polypeptin. In one embodiment, the lipopeptide of the present application is surfactin. In one embodiment, the lipopeptide of the present application is iturin. In one embodiment, the lipopeptide of the present application is fengycin. In one embodiment, the lipopeptide of the present application is a linear product formed by opening the ring of cyclic lipopeptide of the present application.

In one aspect, the lipopeptide involved in the present application is produced by bacteria, actinomycetes, fungi, and cyanobacteria. In one embodiment, the lipopeptide of the present application is produced by bacteria. In one embodiment, the bacteria are wild-type bacteria or genetically engineered bacteria. In one embodiment, the bacteria are selected from the group consisting of Bacillus, Pseudomonas, Streptomyces, Arthrobacter and Burkholderia. In one embodiment, the lipopeptide of the present application is produced by Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus lichenformis, Bacillus megaterium, Bacillus pumilus, Bacillus stearothermophilus, Bacillus subtilis or Bacillus thuringiensis. In one embodiment, the lipopeptide of the present application is produced by Pseudomonas aeruginosa, Pseudomonas fluorescens, Pseudomonas mallei or Pseudomonas pseudomallei. In one embodiment, the lipopeptide of the present application is produced by Streptomyces achromogenes, Streptomyces avermitilis, Streptomyces coelicolor, Streptomyces griseus or Streptomyces lividans. In one embodiment, the lipopeptide of the present application is produced by Arthrobacter globiformis or Arthrobacter luteus. In one embodiment, the lipopeptide of the present application is produced by actinomycetes. In one embodiment, the actinomycetes are selected from the group consisting of: Streptomyces, Amycolatopsis and Microbacterium. In one embodiment, the lipopeptide of the present application is produced by fungi. In one embodiment, the fungi are selected from the group consisting of: Aspergillus, Microascus, Stachylidium, Myrothecium, Sarocladium, Ophiosphaerella and Clavariopsis. In one embodiment, the lipopeptide of the present application is produced by cyanobacteria. In one embodiment, the cyanobacteria are selected from the group consisting of: Okeania, Cladonia and Moorea.

In one embodiment, the lipopeptide of the present application is obtained by cultivating Bacillus or Pseudomonas. In one embodiment, the lipopeptide of the present application is obtained by cultivating Bacillus and regulating the expression of genes related to lipopeptide synthesis. In one embodiment, the genes related to lipopeptide synthesis are selected from the group consisting of: lipopeptide synthesis genes, transmembrane transport protein gene ycxA, biotin carboxylase gene yngH, spore synthesis genes spoIVA/B/C/F, spoVA/B/D/E, and leucine synthesis pathway genes leuABCD/ilvK. In one embodiment, the lipopeptide synthesis genes are selected from the group consisting of: surfactin synthesis gene srfA, fengycin synthesis gene fen, lichenysin synthesis gene lic, bacillomycin synthesis gene bam, mycosubtilin myc, iturin synthesis gene itu, plipastatin synthesis gene pps, fusaricidin fus, kurstakin krs, polymyxin pmx and octapeptin oct. In one embodiment, regulating the expression of genes related to lipopeptide synthesis is selected from the group consisting of: overexpressing lipopeptide synthesis genes (e.g., srfA gene), overexpressing ycxA, overexpressing yngH, knocking out spoIVA/B/C/F, knocking out spoV A/B/D/E, overexpressing leuABCD and overexpressing ilvK. In one embodiment, overexpressing srfA gene is obtained by replacing natural promotor PsrfA with Pg3 promotor. In one embodiment, the Bacillus is Bacillus subtilis THY-7/Pg3-srfA.

### 3. Compositions of the Present Application

In one aspect, the present application relates to a composition for promoting root nodule growth, comprising: (i) 0.001-100 g/L of lipopeptide; and (ii) an adjuvant. In one embodiment, the adjuvant is selected from the group consisting of: solvents, dispersants, wetting agents, polymers and warning colorants. In one embodiment, the adjuvant is water. In one embodiment, the composition comprises 0.001 g/L, 0.002 g/L, 0.003 g/L, 0.004 g/L, 0.005 g/L, 0.006 g/L, 0.007 g/L, 0.008 g/L, 0.009 g/L, 0.01 g/L, 0.02 g/L, 0.03 g/L, 0.04 g/L, 0.05 g/L, 0.06 g/L, 0.07 g/L, 0.08 g/L, 0.09 g/L, 0.1 g/L, 0.2 g/L, 0.3 g/L, 0.4 g/L, 0.5 g/L, 0.6 g/L, 0.7 g/L, 0.8 g/L, 0.9 g/L, 1 g/L, 2 g/L, 3 g/L, 4 g/L, 5 g/L, 6 g/L, 7 g/L, 8 g/L, 9 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L or 100 g/L of the lipopeptide. In one embodiment, the composition comprises 0.001-0.005 g/L, 0.001-0.01 g/L, 0.001-0.05 g/L, 0.001-0.1 g/L, 0.001-0.5 g/L, 0.001-1.0 g/L, 0.001-1.5 g/L, 0.001-2.5 g/L, 0.001-5 g/L, 0.001-8 g/L, 0.001-12 g/L, 0.001-18 g/L, 0.001-20 g/L, 0.001-30 g/L, 0.001-40 g/L, 0.001-50 g/L, 0.001-60 g/L, 0.001-70 g/L, 0.001-80 g/L or 0.001-90 g/L of the lipopeptide. In one embodiment, the composition of the present application further comprises 0.01-99.99 wt % of one or more components selected from the group consisting of: polysaccharides, oligosaccharides, dextrins, amino acids, oligopeptides, proteins, lipoids, fatty acids or the derivatives thereof, inorganic salts, dispersants, wetting agents, polymers, and warning colorants. In one embodiment, the polysaccharides are selected from the group consisting of: peptidoglycan, cellulose, glycogen, starch, chitin, fructan, galactan, and glycosaminoglycan. In one embodiment, the amino acids are common α-amino acids. In one embodiment, the amino acids are selected from the group consisting of: alanine, aspartic acid, arginine, glutamic acid, cysteine, glycine, serine, phenylalanine, histidine, isoleucine, lysine, leucine, methionine, asparagine, proline, glutamine, tryptophan, threonine, valine, and tyrosine. In one embodiment, the oligopeptides are peptides consisting of 2-10 amino acids, peptides consisting of 2-8 amino acids, peptides consisting of 2-6 amino acids. In one embodiment, the oligopeptides are selected from the group consisting of oligopeptide-3, oligopeptide-4, oligopeptide-5 and oligopeptide-6. In one embodiment, the lipoids are selected from the group consisting of: lipoid compounds, phospholipids, glycolipids, cholesterol and the esters thereof. In one embodiment, the fatty acids or the derivatives thereof are selected from the group consisting of: stearic acid, animal fats, and vegetable oils. In one embodiment, the inorganic salts are selected from the group consisting of: sulfates, hydrophosphate, dihydrogen phosphates, phosphates, nitrates, and superphosphates. In one embodiment, the inorganic salts are selected from the group consisting of: sodium salts, potassium salts, calcium salts, iron salts, and ammonium salts. In one embodiment, the inorganic salts are selected from the group consisting of: Na₂SO₄, K₂SO₄, (NH₄)₂SO₄, KH₂PO₄, K₂HPO₄, Na₂HPO₄, NaH₂PO₄, K₃PO₄, CO(NH₂)₂, NH₄NO₃, NaNO₃, CaCl₂, FeCl₂, NH₄Cl, MnSO₄, FeSO₄, Ca(H₂PO₄)₂ and the hydrate thereof. In one embodiment, the dispersants are selected from the group consisting of: naphthalene sulfonate, lignin sulfonate, polycarboxylate and polycarboxylate ester, starch, bovine serum albumin, cyclodextrin. In one embodiment, the wetting agents are selected from the group consisting of: dodecyl sulfate, octyl succinate, alcohol ether sulfate. In one embodiment, the polymers are selected from the group consisting of: polyethylene glycol, polyacrylic emulsion, polyacrylic acid polyvinyl alcohol emulsion, xanthan gum, polyglutamic acid. In one embodiment, the warning colorants are selected from the group consisting of: carmine or fruit green. In one embodiment, the composition of the present application is a culture solution containing 0.001-100 g/L of lipopeptide, which is obtained by cultivating bacteria capable of producing lipopeptides. In one embodiment, the composition of the present application is a solution or solid mixture containing 0.001-100 g/L of lipopeptide, which is formulated from purified or partially purified lipopeptide. In one embodiment, the composition of the present application is aqueous solution, concentrate or dry powder formulations containing lipopeptide.

In one embodiment, the root nodule described herein is produced by bacteria, actinomycetes, fungi, or cyanobacteria that are symbiotic with plants. In one embodiment, the root nodule described herein is produced by bacteria that are symbiotic with plants. In one embodiment, preferably the bacteria are selected from α-, β-, or γ-Proteobacteria, and more preferably are selected from the group consisting of: Rhizobium, Sinorhizobium, Ensifer, Shinella, Neorhizobium, Pararhizobium, Mesorhizobium, Bradyrhizobium, Phyllobacterium, Methylobacterium, Microvirga, Ocrhobactrum, Azorhizobium, Devosia, Burkholderia, Cupriavidu and Pseudomonas. In one embodiment, the the root nodule described herein is produced by actinomycetes that are symbiotic with plants. In one embodiment, preferably the actinomycetes are Frankia, and more preferably selected from the group consisting of: F. alni, F. casuarinae and F. canadensis. In one embodiment, the the root nodule described herein is produced by fungi that are symbiotic with plants. In one embodiment, the fungi are selected from the group consisting of: Ascomycetes and Basidiomycetes. In one embodiment, the root nodule described herein is produced by cyanobacteria that are symbiotic with plants. In one embodiment, the cyanobacteria are selected from the group consisting of: Candida, Anabaena, Scytonema and Stigonema.

In one embodiment, the root nodule described herein is produced by bacteria that are symbiotic with plants. In one embodiment, the plants are leguminous or cannabis family of plants. In one embodiment, preferably the plants are selected from the group consisting of: soybeans, broad beans, peas, mung beans, adzuki beans, cowpeas, kidney beans, lentils, winged beans, black beans, chickpeas, peanuts, purple vetch, alfalfa, Chinese milk vetch, acacia, Dalbergia hupeana Hance, Chinese honey locust, Erythrophleum fordii Oliv., Sophora japonica, Indigofera pseudotinctoria, Indigofera tinctoria L, sappan wood, arabic tree gum, Astragalus membranaceus, copal gum tree, Crotalaria juncea L., kudzu vine, Trema tomentosa, and Parasponia.

In one embodiment, the root nodule described herein is produced by Frankia or cyanobacteria that are symbiotic with plants. In one embodiment, the plants are selected from the group consisting of: Betulaceae, Casuarinaceae, Myricaceae, Rhamnaceae, Elaeagnaceae, Datiscaceae, Coriariaceae, Rosaceae, Bryophytes, ferns and cycades. In one embodiment, the root nodule described herein is produced by fungi that are symbiotic with plants. In one embodiment, the plants are algaes. In one embodiment, the algaes are selected from the group consisting of: Nostoc, Coccidium, and Trentepohlia.

### 4. Methods for Preparing the Composition of the Present Application

In one aspect, the present application relates to a method for preparing the composition for promoting root nodule growth described herein. In one embodiment, the method comprises: cultivating the bacteria capable of producing lipopeptides under conditions suitable for lipopeptide production to obtain a culture solution containing 0.001-100 g/L of lipopeptides, and using the culture solution as the composition herein; or formulating purified or partially purified lipopeptides into solutions or solid mixtures containing 0.001-100 g/L of lipopeptides, and using the solutions or solid mixtures as the composition herein. In one embodiment, the solutions are aqueous solutions. In one embodiment, the solvent in the aqueous solutions is deionized water or tap water. In one embodiment, the method further comprises: to the culture solution, solutions or solid mixtures, adding 0.01-99.99 wt % of one or more components selected from the group consisting of: polysaccharides, oligosaccharides, dextrins, amino acids, oligopeptides, proteins, lipoids, fatty acids or the derivatives thereof, inorganic salts, dispersants, wetting agents, polymers, and warning colorants; and using the resulting product as the composition herein. In one embodiment, the method further comprises: concentrating and/or drying and/or partially purifying the culture solution, solution, solid mixture, or product, and using the concentrated and/or dried and/or partially purified product as the composition herein.

### 5. Methods for Promoting Root Nodule Growth of the Present Application

In one aspect, the present application relates to a method for promoting root nodule growth by using the compositions herein. In one embodiment, the method comprises: (1) directly applying the composition to the culture of the plants that are symbiotic with bacteria, actinomycetes, fungi, or cyanobacteria which produce the root nodule, the culture is preferably soil; (2) cultivating and harvesting the plants; (3) optionally measuring the parameters of the root nodule, and comparing them with those of the control group to which no composition is applied. In one embodiment, the parameters are selected from the group consisting of: the number of root nodules and root nodule weight of each plant. In one embodiment, the promotion of root nodule growth is characterized by one or more features selected from the group consisting of: (1) the number of root nodules per plant of the plants cultivated by applying the composition is at least 1.1 times, at least 1.2 times, at least 1.3 times, at least 1.4 times, at least 1.5 times, at least 1.6 times, at least 1.7 times, at least 1.8 times, at least 1.9 times, at least 2 times, at least 2.5 times, at least 3 times, at least 3.5 times, at least 4 times, at least 4.5 times, more preferably at least 5 times, at least 5.5 times, at least 6 times, at least 6.5 times, at least 6.6 times, at least 6.7 times, at least 6.8 times, at least 6.9 times, more preferably at least 7 times that of the control group to which no composition is applied; and/or (2) the root nodule weight per plant of the plants cultivated by applying the composition is at least 1.1 times, at least 1.2 times, at least 1.3 times, at least 1.4 times, at least 1.5 times, at least 1.6 times, at least 1.7 times, at least 1.8 times, at least 1.9 times, at least 2 times, at least 2.5 times, at least 3 times, at least 3.5 times, at least 4 times, at least 4.5 times, more preferably at least 5 times, at least 5.5 times, at least 6 times, at least 6.5 times, at least 6.6 times, at least 6.7 times, at least 6.8 times, at least 6.9 times, more preferably at least 7 times that of the control group to which no composition is applied.

In one embodiment, the composition herein can promote root nodule growth and meanwhile can promote the growth of the plants that are symbiotic with bacteria, actinomycetes, fungi, or cyanobacteria which produce the root nodule. In one embodiment, the promotion of the growth of the plants is characterized by the parameters selected from the group consisting of: the plant height of the aboveground part, the fresh weight of the aboveground part, the fresh weight of the underground part, and the number or weight of plant products. In one embodiment, the promotion of the growth of the plants is characterized by one or more features selected from the group consisting of: (1) the plant height of the aboveground part of the plants cultivated by applying the composition increases 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, more preferably 36% or more, as compared to the control group; (2) the fresh weight of the aboveground part of the plants cultivated by applying the composition increases 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, preferably 45% or more, more preferably 47% or more, as compared to the control group; (3) the fresh weight of the underground part of the plants cultivated by applying the composition increases 3% or more, 4% or more, 5% or more, 6% or more, 7% or more, 8% or more, 9% or more, 10% or more, 11% or more, more preferably 12% or more, as compared to the control group; and/or (4) the number or weight of plant products cultivated by applying the composition is at least 1.1 times, at least 1.2 times, at least 1.3 times, at least 1.4 times, at least 1.5 times, at least 1.6 times, at least 1.7 times, at least 1.8 times, at least 1.9 times, at least 2.0 times, at least 2.1 times, at least 2.2 times, at least 2.3 times, at least 2.5 times, preferably at least 3.0 times that of the control group. In one embodiment, the plant products are selected from a group consisting of: fruit pods, pegs and seeds of the plant.

In one embodiment, the application herein is selected from a group consisting of: seed coating, seed soaking treatment, bulb treatment, root irrigation treatment, drip irrigation treatment, and foliar spraying application.

### 6. Uses of the Present Application

In one aspect, the present application relates to use of the composition or lipopeptide herein for promoting root nodule growth. In one embodiment, the composition or lipopeptide herein is used for promoting root nodule growth and meanwhile promoting the growth of the plants that are symbiotic with bacteria, actinomycetes, fungi, or cyanobacteria which produce the root nodule. In one embodiment, provided herein is use of the composition or lipopeptide herein in agricultural fertilizers, agricultural adjuvants or pesticides. In one embodiment, the fertilizers are water-soluble fertilizers containing amino acid.

In one embodiment, the present application relates to use of a composition for promoting root nodule growth, wherein the composition comprises: (i) 0.001-100 g/L of lipopeptide; and (ii) an adjuvant, wherein the lipopeptide is one or more selected from a group consisting of surfactin family, iturin family and fengycin family, wherein the root nodule is produced by bacteria, actinomycetes, fungi, or cyanobacteria that are symbiotic with plants, and the plants are selected from: leguminous plants, Betulaceae, Casuarinaceae, Myricaceae, Rhamnaceae, Elaeagnaceae, Coriariaceae, Rosaceae and Cycadaceae plants.

In one embodiment, the present application relates to use of lipopeptide for promoting root nodule growth, wherein the lipopeptide is one or more selected from a group consisting of surfactin, iturin and fengycin, wherein the root nodule is produced by bacteria, actinomycetes, fungi, or cyanobacteria that are symbiotic with plants, and the plants are selected from: leguminous, Betulaceae, Casuarinaceae, Myricaceae, Rhamnaceae, Elaeagnaceae, Coriariaceae, Rosaceae and Cycadaceae plants.

In the present application, when using lipopeptides to promote root nodule growth, its concentration used is typically 0.001-100 g/L. In other words, as long as lipopeptides can be supplied to the root nodules at a concentration of 0.001-100 g/L, there are no limitations on the form in which they are applied.

### 7. Advantages of the Present Application

The present application has the following technical advantages:

(1) The compositions or lipopeptides herein can significantly promote root nodule growth, and can increase the number of nodules, enlarge the volume of nodules, and increase the weight of nodules. Furthermore, compared with the control, the aboveground and underground parts of plants treated with the composition are more developed, and the number and/or yield of plant products are higher.

(2) The lipopeptides used in the compositions herein are biodegradable, pollution-free, environmentally friendly, and harmless when used over long periods of time.

(3) The lipopeptides used in the compositions herein are highly stable, less affected by temperature and humidity, easy to prepare and use, and convenient to manage.

### DETAIL DESCRIPTION OF THE INVENTION

Now the present application will be further illustrated with reference to specific examples, but these specific examples cannot be understood as a limitation on the scope of the present application. Those skilled in the art may make a variety of changes or modifications to these specific examples without departing from the technical solutions of the present application, and the embodiments after such changes or modifications still fall within the scope of the present application.

### 4.1 Equipment, Materials, and Reagents

Ultra-clean workbench, shakers, incubator, shaking flask, carbohydrate, inorganic nitrogen sources, organic nitrogen sources, vegetable oil, KH₂PO₄, Na₂HPO₄·12H₂O, CaCl₂, MnSO₄·H₂O, FeSO₄·7H₂O are all commercial available products.

### Strain

Bacillus subtilis THY-7/Pg3-srfA was obtained from the patent ZL201510654218.3; THY-7/Pg3-srfA(yngH) was obtained from the patent ZL2018108652957; Bacillus subtilis THY-7/Pg3-srfAΔspoIVA, THY-7/Pg3-srfAΔspoIVB, THY-7/Pg3-srfAΔspoIVC, THY-7/Pg3-srfAΔspoIVF, THY-7/Pg3-srfAΔspoVA, THY-7/Pg3-srfAΔspoVB, THY-7/Pg3-srfAΔspoVD and THY-7/Pg3-srfAΔspoVE were obtained from the patent ZL 201811465067.7; Bacillus subtilis THY-7/Pg3-srfA(leuABCD-ilvK) was obtained from the patent ZL 201910549289.5.

### Example 1. Obtaining Lipopeptide Compositions

### 1. Obtaining Compositions Containing Surfactin

A single colony of Bacillus subtilis THY-7/Pg3-srfA was selected and picked, and then was incolated into a LB liquid medium. The colony was incubated at 37° C. and 200 rpm for 16 hours to obtain the seed culture. The seed culture was added and incolated at a ratio of 5% to a shaking flask containing the fermentation medium. When cultivating under the condition of 37° C. and 200 rpm for 2-6 hrs, 1 mM IPTG was added. And a fermentation broth containing surfactin was obtained after a continuous incubation for 2 days.

The used fermentation medium had the following composition: 30-100 g/L of carbohydrates, 10-50 g/L of inorganic nitrogen source, 0.5-3 g/L of organic nitrogen source, 0.1-1 g/L of KH₂PO₄, 0.5-0.3 g/L of Na₂HPO₄·12H₂O, 0.002-0.01 g/L of CaCl₂, 0.002-0.01 g/L of MnSO₄·H₂O, 0.002-0.01 g/L of FeSO₄·7H₂O, pH 6.5-7.5, 0.01-20 wt % of fermentation additives (silica-based defoamers, polysaccharides, amino acids or the derivatives thereof, oligopeptides, phospholipids, glycolipids, and/or fatty acids or the derivatives thereof).

The detection of surfactin content in fermentation broth was performed using the method disclosed in CN105400784A. Specifically, it was: 1 mL of fermentation broth was centrifuged at 12,000 rpm for 1 minute, 100 μL of the supernatant was taken from the broth and then added to 1,900 μL of deionized water, mixed, filtered through a 0.22 μm filter membrane, and analyzed by HPLC. The mobile phase used in HPLC analysis consisted of methanol and water in a ratio of 85/15, the flow rate was 1 mL/min, the chromatographic column was a C18-ODS reversed-phase chromatographic column, and the column temperature was 40° C., with a UV detector set to a detection wavelength of 205 nm.

It can be known by testing that, the content of surfactin in the fermentation broth is approximately 0.001-90 g/L. The fermentation broth is Composition 1 containing surfactin as described herein. The surfactin was extracted from the fermentation broth and obtained as a powder product through freeze-drying (which can also be directly applied as the composition herein), and the powder was formulated into an aqueous solution with a concentration of 0.001-100 g/L, which was used as Composition 2 described herein.

### 2. Obtaining Compositions Containing Iturin and Fengycin

Iturin and fengycin were purchased from MedChemExpress. They were dissolved in water, so as to be formulated into an aqueous solution with a concentration of 0.001-100 g/L, which were used as Compositions 3 and 4 desribed herein.

Into Compositions 2-4 was added 0.01-99.99 wt % of polysaccharides or the derivatives thereof (one of more of peptidoglycan, cellulose, glycogen, starch, chitin, polyfructose, polygalactose, and glycosaminoglycans), amino acids (common α-amino acids), oligopeptides (one of more of oligopeptide-3, oligopeptide-4, oligopeptide-5, and oligopeptide-6), lipoids (one of more of ester compounds, phospholipids, glycolipids, and cholesterol and the esters thereof), fatty acids or the derivatives thereof (one of more of stearic acid, animal fats, and plant oils), inorganic salts (one of more of Na₂SO₄, K₂SO₄, (NH₄)₂SO₄, KH₂PO₄, K₂HPO₄, Na₂HPO₄, NaH₂PO₄, K₃PO₄, CO(NH₂)₂, NH₄NO₃, NaNO₃, CaCl₂, FeCl₂, NH₄Cl, MnSO₄, FeSO₄, Ca(H₂PO₄)₂), dispersants (one of more of naphthalene sulfonates, lignosulfonates, polycarboxylates and polycarboxylate esters, starch, bovine serum albumin, cyclodextrin), wetting agents (one of more of dodecyl sulfate, octyl succinate, alcohol ether sulfate polymers), polymers (one of more of polyethylene glycol, polyacrylic emulsion, polyacrylic acid polyvinyl alcohol emulsion, xanthan gum, polyglutamic acid), or warning colorants, as Compositions 5-7 herein (see Table 1) (the aforesaid Compositions 1-7 may also be applied directly in the form of powder mixtures as the compositions herein).

TABLE 1

| | Content of added components in different | | |
|---|---|---|---|
| | compositions (wt %) | | |
| | Compo- | Compo- | Compo- |
| Added components | sition 5 | sition 6 | sition 7 |
| cellulose | 0.1-0.5 | 0.01-0.03 | 0.03-0.1 |
| chitosan | 0.01-0.05 | 0.05-4 | 0.05-4 |
| glutamic acid | 0.1-0.8 | 0.2-1.0 | 0.1-1.0 |
| methionine | 0.1-0.1 | 0.01-0.05 | 0.01-0.03 |
| cysteine | 0.01-0.03 | 0.01-0.05 | 0.01-0.05 |
| oligopeptide-6 | 0.01-1 | 0.01-1 | 0.01-1 |
| oligopeptide-3 | 0.01-0.03 | 0.03-0.03 | 0.3-1 |
| phospholipid | 0.01-0.1 | 0.01-0.1 | 0.01-0.1 |
| glycolipid | 0.01-0.1 | 0.01-0.1 | 0.01-0.1 |
| ethylene glycol stearate | 0.01-0.5 | 0.01-0.5 | 0.01-0.5 |
| stearic acid | 0.1-5 | 0.1-5 | 0.1-5 |
| soybean oil | 0.1-10 | 0.1-10 | 0.1-10 |
| sodium sulfate | 1-50 | 10-60 | 1-50 |
| silica | 1-50 | 1-50 | 1-50 |
| urea | 1-20 | 1-20 | 1-20 |
| potassium dihydrogen | 1-10 | 0.1-40 | 0.1-40 |
| phosphate | | | |
| dibasic potassium phosphate | 1-10 | 0.1-40 | 0.1-40 |
| potassium phosphate | 1-10 | 0.1-40 | 0.1-40 |
| ammonium nitrate | 1-10 | 0.1-40 | 0.1-40 |
| calcium superphosphate | 1-10 | 0.1-40 | 0.1-40 |
| naphthalene sulfonate | 0.1-40 | 0.1-40 | 0.1-40 |
| lignosulfonate | 1-40 | 1-40 | 1-40 |
| starch | 1-40 | 1-40 | 1-40 |
| dodecyl sulfonate | 0.1-20 | 0.1-20 | 0.1-20 |
| alcohol ether sulfate | 0.1-20 | 0.1-20 | 0.1-20 |
| polyethylene glycol | 1-30 | 1-30 | 1-30 |
| polyacrylic emulsion | 1-30 | 1-30 | 1-30 |
| xanthan gum | 1-30 | 1-30 | 1-30 |
| polyglutamic acid | 1-30 | 1-30 | 1-30 |
| carmine | 0-5 | 0-5 | 0-5 |
| fruit green | 0-5 | 0-5 | 0-5 |

### Example 2. Lipopeptide Compositions Promote the Growth of Root Nodules and Plants of Soybeans

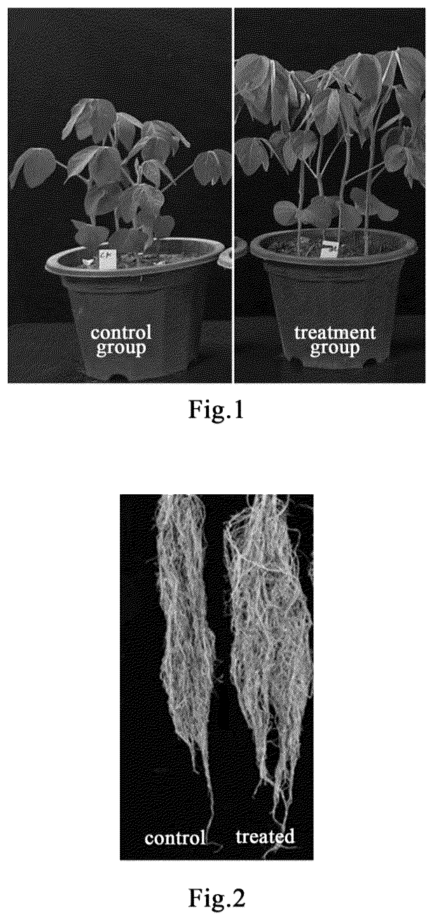

After irrigating the soil-filled flowerpots, six soybeans were planted per flowerpot. At post-emergence stage, the plants were thinned to 4 plants/flowerpot. Within 2 weeks of the post-emergence stage, 1-200 mL of lipopeptide Compositions 1-7 were irrigated. The growth situation of the aboveground parts of the potted plants 15 days after treatment, and the height of the plants were measured. Measurements showed that the average plant height in the treatment groups was 21-25 cm (with an average height of 21 cm in the treatment group treated with Composition 1, an average height of 23 cm in the treatment group treated with Composition 2, an average height of 22 cm in the treatment group treated with Composition 3, an average height of 22 cm in the treatment group treated with Composition 4, an average height of 25 cm in the treatment group treated with Composition 5, an average height of 24.3 cm in the treatment group treated with Composition 6, an average height of 24 cm in the treatment group treated with Composition 7), while the average height of the plants in the control group was 17.9 cm. It can be seen that the treatment groups treated with the compositions showed an increase of 17.3-39.7% compared to the control group (see FIG. 1, which is the treatment group treated with Composition 2). After 25 days of treatment, the entire plant was removed from the soil, and the aboveground and underground parts of the plant were separated. The aboveground part was weighed directly to determine its fresh weight. Upon measurement, the fresh weight of the aboveground parts in the treatment groups ranged from 33-40.1 g (with an average fresh weight of 35 g in the treatment group treated with Composition 1, an average fresh weight of 37 g in the treatment group treated with Composition 2, an average fresh weight of 33 g in the treatment group treated with Composition 3, an average fresh weight of 34 g in the treatment group treated with Composition 4, an average fresh weight of 40.1 g in the treatment group treated with Composition 5, an average fresh weight of 38 g in the treatment group treated with Composition 6, an average fresh weight of 39 g in the treatment group treated with Composition 7), while the fresh weight of the aboveground part in the control group was 27.2 g. The treatment groups treated with the compositions showed an increase of 21.3-47.4% compared to the control group. After rinsing and cleaning the underground parts with tap water, the underground parts were weighed, and the weight was used as the fresh weight of the underground parts. Upon measurement, the fresh weight of the underground parts in the treatment groups treated with the compositions ranged from 24.5-29.1 g (with the average fresh weights of 25 g, 28 g, 24.5 g, 29.1 g, 29 g, 27.3 g, 28.0 g in the treatment groups treated with Compositions 1-7, respectively), while the fresh weight of the underground parts in the control group was 21.6 g. The treatment groups treated with the compositions showed an increase of 13.4-34.7% compared to the control group (see FIG. 2, which is the treatment group treated with Composition 2).

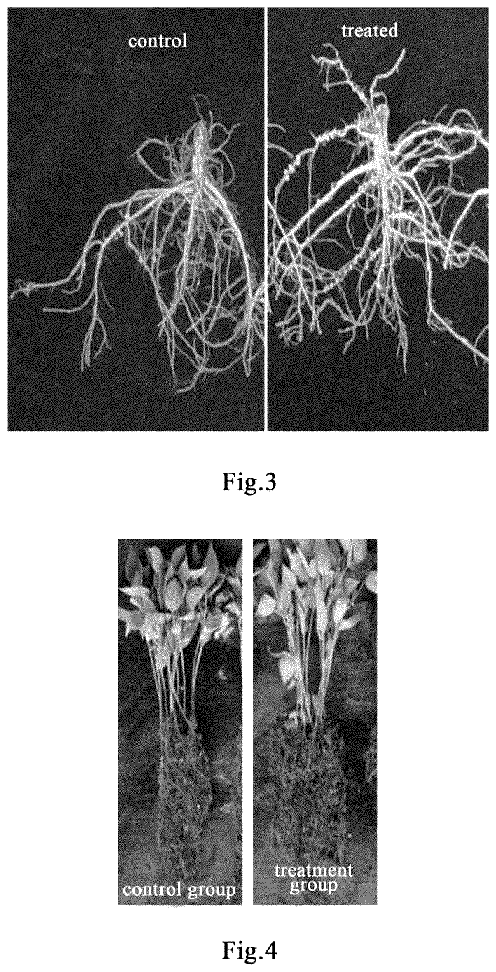

Soybeans were sown in the field, and within 2 weeks after sowing, 1-200 mL of lipopeptide Composition 1-7 were applied via irrigating. After irrigating application for 3 weeks, the growth of the soybean plants was observed, and the plant height was measured. The control group had a plant height of approximately 51 cm, while the treatment group treated with lipopeptide had an average plant height of approximately 57-60 cm (wherein each treatment group treated with Compositions 1-7 had an average plant height of 57 cm, 58.8 cm, 57.3 cm, 57.5 cm, 60 cm, 59 cm, and 59.5 cm, respectively), representing an increase of 11.8-17.6% compared to the control group. After 8 weeks of sowing, the entire plant was removed from the soil, and the aboveground and underground parts of the plant were separated and measured. The fresh weight of the aboveground part of the control group was 295 g, while the fresh weights of the aboveground parts of the treatment groups treated with the compositions ranged from 350 to 500 g (wherein each treatment group treated with Compositions 1-7 had an average fresh weight of 405 g, 420 g, 350 g, 363 g, 500 g, 495 g, and 407 g, respectively), representing an increase of 18.6-69.5% compared to the control group. After rinsing and cleaning the underground parts with tap water, the underground parts were weighed, and the weight was used as the fresh weight of the underground parts. The fresh weight of the underground parts in the treatment groups ranged from 27.5 to 33.6 g (wherein each treatment group treated with Compositions 1-7 had an average fresh weight of 31 g, 29.8 g, 28.7 g, 27.5 g, 33.6 g, 33 g, and 32 g, respectively), while the control group had an average fresh weight of 23.8 g. The treatment groups showed an increase of 15.5-41.2% compared to the control group. Upon measurement, the average number of root nodules in the treatment group treated with the compositions was 43.1 (wherein each treatment group treated with Compositions 1-7 had an average number of root nodules of 40, 39, 42, 38, 51, 45, and 47, respectively), while the control group had an average number of root nodules of 6.1. Thus, the treatment group treated with the compositions had an average number of root nodules of approximately 7 times that of the control group. After the root nodules were removed and weighed, the average root nodule weight of the treatment group was approximately 10.3 times that of the control group (see FIG. 3, which is the treatment group treated with Composition 5).

The soybeans were harvested, washed and dried in the sun. After that, the yield was determined. The soybean yield of the treatment groups showed an increase of 23-40% compared to the control group (wherein each treatment group treated with Compositions 1-7 had an increase in average yiled of 37%, 38%, 23%, 29%, 40%, 39%, 39%, respectively).

After seed-dressing soybeans with 1-100 mL of Composition 1-7, they were sowed in flowerpots filled with soil, with 6 plants per flowerpot, and were irrigated. At post-emergence stage, the plants were thinned to 4 plants/flowerpot. They were irrigated and cared normally. One month after sowing, the entire plant was removed from the flowerpot, the aboveground and underground parts of the plant were separated and measured. The fresh weight of the aboveground part of the control group was 28.9 g, while the fresh weights of the aboveground parts of the treatment groups treated with the compositions ranged from 35.3 to 39.1 g (wherein each treatment group treated with Compositions 1-7 had an average fresh weight of 36 g, 36.9 g, 35.3 g, 36.1 g, 39.1 g, 38.2 g, 37 g, respectively), representing an increase of 22.1-35.3% compared to the control group. After rinsing and cleaning the underground parts with tap water, the underground parts were weighed, and the weight was used as the fresh weight of the underground parts. The fresh weight of the underground parts in the treatment groups ranged from 25.7 to 30.2 g (wherein each treatment group treated with Compositions 1-7 had an average fresh weight of underground part of 27 g, 25.7 g, 26 g, 28 g, 30.2 g, 30.1 g, 29 g, respectively), while the control group had an average fresh weight of underground part of 23.0 g. The treatment groups showed an increase of 11.7-31.3% compared to the control group. Upon measurement, the average number of root nodules in the treatment group treated with the compositions was 36.3 (wherein each treatment group treated with Compositions 1-7 had an average number of root nodules of 33, 35, 34, 35, 41, 37, 39, respectively), which was 6.05 times that of the control group. After the root nodules were removed and weighed, the average root nodule weight of the treatment group was approximately 5.9 times that of the control group.

### Example 3. Lipopeptide Compositions Promote the Growth of Root Nodules and Plants of Mung Beans

Mung beans were sown in the field, and within 2 weeks post-emergence, 1-200 mL of lipopeptide Composition 1-7 were applied via irrigating. After irrigating application for 3 weeks, the growth of the mung bean plants was observed, and the plant height was measured. Upon measurement, the plants in each treatment group had an average plant height of approximately 13-16 cm (wherein each treatment group treated with Compositions 1-7 had an average plant height of 13.4 cm, 13.9 cm, 13.0 cm, 15 cm, 16 cm, 14.3 cm, 15.1 cm, respectively), while the control group had an average plant height of 11.9 cm. It can be seen that the treatment groups treated with the compositions showed an increase of 11.0-34.5% compared to the control group (see FIG. 4, which is the treatment group treated with Composition 4).

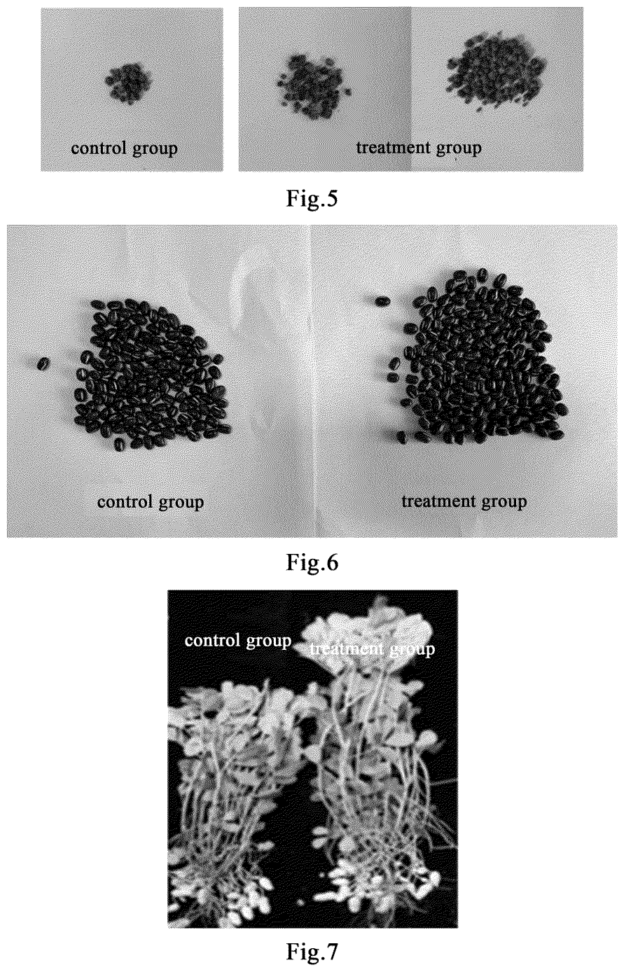

After 10 weeks of treatment, the entire plant was removed from the soil, and the underground parts were rinsed with tap water. As shown in FIG. 4, upon measurement, the fresh weight of the underground parts in the treatment group ranged from 34.8 to 39.5 g (wherein each treatment group treated with Compositions 1-7 had an average fresh weight of underground part of 35.1 g, 34.8 g, 34 g, 36 g, 39.5 g, 37.2 g, 38.8 g, respectively), while the control group had an average fresh weight of underground part of 27.84 g. The treatment groups showed an increase of 25-42% compared to the control group. Furthermore, the number and weight of root nodules in the underground parts were measured (see FIG. 5, which is the treatment group treated with Composition 5). The average number of root nodules in the treatment groups was 88-124 (wherein each treatment group treated with Compositions 1-7 had an average number of root nodules of 95, 99, 93, 88, 123, 105, and 124, respectively), while the control group had an average number of root nodules of 50. The treatment group showed an increase of 76%-148% compared to the control group. After the root nodules were removed and weighed, the average root nodule weight of the treatment group was 2.5-4 times that of the control group, as shown in FIG. 5.

The mung beans were harvested, washed and dried in the sun. After that, the yield was determined. The mung bean yield of the treatment groups showed an increase of 18-35% compared to the control group (wherein each treatment group treated with Compositions 1-7 had an increase in average yiled of 32%, 30%, 18%, 23%, 35%, 33%, 35%, respectively), and the plumpy kernel rate of the treatment groups showed an increase of 8-15% compared to the control group.

Mung beans were sown in the field. When emerging to reach four-leaf stage, the plants were applied 1-200 mL of Lipopeptide Composition 1-7 via foliar spray. After 3 weeks, the growth of the mung bean plants was observed, and the plant height was measured. Upon measurement, the plants in each treatment group had an average plant height of 12.6-15.1 cm (wherein each treatment group treated with Compositions 1-7 had an average plant height of 12.6 cm, 13.0 cm, 12.8 cm, 13.1 cm, 15.1 cm, 14.2 cm, and 15.0 cm, respectively), while the control group had an average plant height of 11.9 cm. It can be seen that the treatment groups treated with the compositions showed an increase of 5.9-26.9% compared to the control group. After 10 weeks, the entire plant was removed from the soil, and the underground parts were rinsed with tap water, and the fresh weight of the underground part, the number of root nodules and the root nodule weight were measured. Upon measurement, the fresh weight of the underground parts in the treatment groups ranged from 31.5-35.3 g (wherein each treatment group treated with Compositions 1-7 had an average fresh weight of underground part of 32.0 g, 31.5 g, 31 g, 32.3 g, 35.3 g, 34.1 g, and 35.3 g, respectively), while the control group had an average fresh weight of underground part of 27.84 g. The treatment group treated with the compositions showed an increase of 13.1-26.8% compared to the control group. The average number of root nodules in the treatment groups was 65-90 (wherein each treatment group treated with Compositions 1-7 had an average number of root nodules of 80, 89, 65, 71, 88, 73, and 90, respectively), while the control group had an average number of root nodules of 50. The treatment group showed an increase of 30%-80% compared to the control group, and the average root nodule weight of the treatment group was 1.7-2.7 times that of the control group.

### Example 4. Lipopeptide Compositions Promote the Growth of Root Nodules and Plants of Adzuki Beans

Adzuki beans were sown in the field, and within 1-2 weeks post-emergence, 1-200 mL of Lipopeptide Composition 1-7 were applied via irrigating. After irrigating application for 3 weeks, the growth of the red bean plants was observed, and the plant height was measured. Upon measurement, the plants in each treatment group had an average plant height of approximately 13.6-18.4 cm (wherein each treatment group treated with Compositions 1-7 had an average plant height of 18.3 cm, 18.2 cm, 17.9 cm, 13.6 cm, 18.4 cm, 18.0 cm, 16.1 cm, respectively), while the control group had an average plant height of 12.2 cm. It can be seen that the treatment groups treated with the compositions showed an increase of 11.5-50.8% compared to the control group.

After 10 weeks of treatment, the entire plant was removed from the soil, and the aboveground and underground parts of the red bean plant were separated and weighed, respectively. Upon measurement, the fresh weight of the aboveground parts in the treatment groups ranged from 30.2-35.5 g (wherein each treatment group treated with Compositions 1-7 had an average fresh weight of the aboveground parts of 35.1 g, 35 g, 31 g, 30.2 g, 35.5 g, 34.9 g and 33 g, respectively), while the fresh weight of the aboveground part in the control group was 25.3 g. The treatment groups treated with the compositions showed an increase of 19.4-4.3% compared to the control group. The fresh weight of the underground parts in the treatment groups ranged from 24.7-28.4 g (wherein each treatment group treated with Compositions 1-7 had an average fresh weight of the underground parts of 28.2 g, 28 g, 25 g, 24.7 g, 28.4 g, 27 g and 27 g, respectively), while the fresh weight of the underground part in the control group was 20.6 g. The treatment groups treated with the compositions showed an increase of 19.9-37.9% compared to the control group. Furthermore, the number of root nodules in the underground parts was also measured. The average number of root nodules in the treatment groups was 63-108 (wherein each treatment group treated with Compositions 1-7 had an average number of root nodules of 99, 100, 85, 63, 108, 103, and 95, respectively), while the control group had an average number of root nodules of 41. The treatment group showed an increase of 53.6%-163.4% compared to the control group. After the root nodules were removed and weighed, the average root nodule weight of the treatment group was 2.1-5 times that of the control group. The adzuki beans were harvested. The treatment groups had an average pod number of 18-20, while the control group had an average pod number of 15; the treatment groups had an average number of adzuki beans of 138-160/plant (wherein each treatment group treated with Compositions 1-7 had an average yield of adzuki beans per plant of 158, 159, 142, 138, 160, 150 and 152, respectively), while the control group had an average yield of adzuki beans per plant of 120. The treatment groups showed an increase of 15-33.3% compared to the control group (see FIG. 6, which is the treatment group treated with Composition 1). The average yield of adzuki beans of the treatment groups increases 17-40% compared to the control group.

### Example 5. Lipopeptide Compositions Promote the Growth of Root Nodules and Plants of Kidney Beans

The kidney bean seedlings were transplanted in the field, and rejuvenated for 1 week. After that, 1-200 mL of lipopeptide Composition 1-7 were applied via irrigating. Frameworks were set up in time. After irrigating for 2 weeks, the vine seedlings in the treatment groups began to clime the frameworks, whereas those in the control group did not begin to pull vine. After a four-week treatment, 1-200 mL of Lipopeptide Composition 1-7 were applied via irrigating once more. After the second four-week treatment, the kidney bean plant and underground root system were observed. Upon measurement, the aboveground fresh weight of the kidney bean plants in the treatment groups were 56-72 g (wherein each treatment group treated with Compositions 1-7 had an average aboveground fresh weight of 63 g, 61 g, 56 g, 61 g, 70 g, 64 g and 72 g, respectively), while that in the control group was 42 g. The treatment groups showed an increase of 33.3-71.4% compared to the control group. The fresh weight of the underground parts in the treatment groups ranged from 40-51 g (wherein each treatment group treated with Compositions 1-7 had an average fresh weight of the underground parts of 43 g, 40 g, 45 g, 45 g, 51 g, 48 g, and 49 g, respectively), while the average fresh weight in the control group was 36 g. The treatment groups showed an increase of 11.1-41.7% compared to the control group. Furthermore, the number of root nodules in the underground parts was also measured. The average number of root nodules in the treatment groups was 74-101 (wherein each treatment group treated with Compositions 1-7 had an average number of root nodules of 80, 82, 85, 74, 83, 100, 93 and 101, respectively), while the control group had an average number of root nodules of 50. The treatment group showed an increase of 48%-102% compared to the control group. After the root nodules were removed and weighed, the average root nodule weight of the treatment group was 1.3-2.1 times that of the control group.

### Example 6. Lipopeptide Compositions Promote the Growth of Root Nodules and Plants of Peanuts

Peanuts were sown in the field and covered with plastic mulch. When the seedlings emerged, the mulch was peeled off in time. After 2 weeks, the mulch was totally removed, and 1-200 mL of lipopeptide Composition 1-7 were applied via irrigating. After irrigating for 6 weeks, the growth of the peanut plants was observed, and the plant height was measured (see FIG. 7, which is the treatment group treated with Composition 2). Upon measurement, the average height of the plants in each treatment groups was 55-60 cm (wherein each treatment group treated with Compositions 1-7 had an average plant height of 56 cm, 59 cm, 55 cm, 55 cm, 60 cm, 57 cm and 57 cm, respectively), while the control group had an average plant height of 50 cm. It can be seen that the treatment groups treated with the compositions showed an increase of 10-20% compared to the control group. The fresh weight of the aboveground parts in the treatment groups ranged from 125-165 g (wherein each treatment group treated with Compositions 1-7 had an average fresh weight of the aboveground parts of 158 g, 155 g, 149 g, 125 g, 164 g, 165 g and 160 g, respectively), while the average fresh weight in the control group was 110 g. The treatment groups treated with the compositions showed an increase of 13.6-50% compared to the control group. The fresh weight of the underground parts in the treatment groups ranged from 75-113 g (wherein each treatment group treated with Compositions 1-7 had an average fresh weight of the underground parts of 78 g, 78 g, 75 g, 75 g, 110 g, 113 g, and 104 g, respectively), while the average fresh weight in the control group was 60 g. The treatment groups treated with the compositions showed an increase of 25-88.3% compared to the control group. Furthermore, the number of root nodules and the peg in the underground parts were also measured. The number of root nodules in the treatment groups was 103-180 (wherein each treatment group treated with Compositions 1-7 had an average number of root nodules of 135, 142, 135, 103, 178, 180 and 170, respectively), while the control group had a number of root nodules of 70. The treatment groups showed an increase of 47.1%-157.1% compared to the control group. After the root nodules were removed and weighed, the average root nodule weight of the treatment groups was 1.8-4.5 times that of the control group. The average peg number per plant in the treatment groups was 10-16 (wherein each treatment group treated with Compositions 1-7 had an average peg number per plant was 14, 14, 12, 10, 16, 15 and 14, respectively), while the control group had an average peg number per plant of 7. The treatment groups showed an increase of 42.9%-128.6% compared to the control group.

After treating for 12 weeks, the peanuts were harvested, washed and dried in the sun. After that, the yield of peanuts was determined. The peanut yield of the treatment groups showed an increase of 20-36.5% compared to the control group (wherein each treatment group treated with Compositions 1-7 had an increase in average yiled of 33%, 35%, 26%, 20%, 36%, 36.5%, 35%, respectively), and the plumpy kernel rate of the treatment groups showed an increase of 10-21% compared to the control group. The platycladi semen weight in the treatment groups was 156.2-170.8 g (wherein each treatment group treated with Compositions 1-7 had an average weight of 168 g, 167 g, 156.2 g, 160 g, 170.8 g, 170 g, 161 g) for 100 peanuts, while the control group had an average weight of 135.1 g for 100 peanuts. The treatment groups showed an increase of 15.6%-26.4% compared to the control group. The weight of 100 kernels of the treatment groups increased 5.3%-28.6% compared to the control group.

### Example 7. Lipopeptide Compositions Promote the Growth of Root Nodules and Plants of Non-Leguminous Plants

7.1 In the field where alder and red birch (Betulaceae), casuarina glauca (Casuarinaceae), bayberry and fragrant fernwood (Myrtaceae), buckthorn (Rhamnaceae), sea-buckthorn and oleaster (Elaeagnaceae Juss.), Coriaria sinica (Coriariaceae), dryas octopetala (Rosaceae), Cycas szechuanensis and Cycas panzhihuaensis (Cycadaceae) have been transplanted for 1-3 months, the clean water was used as a control, and lipopeptide Composition 1-7 were irrigated at 1000-2000 mL per plant monthly. The plants were cultivated for 8-10 months. During the planting process, fertility and water management was uniformly handled by the plantation. The plants were harvested and collected. The underground fresh weight, number of root nodules and root nodule weight were measured for each plant. The data were normalized by using the data of the groups treated with clean water for each plant as standard. The results were shown in Table 2.

TABLE 2

| | The normalization processing results by using the | | |
|---|---|---|---|
| | data of clean water control groups as standard | | |
| | underground | number | root |
| | fresh | of root | nodule |
| Plants | weight % | nodules % | weight % |
| alder | 114-129 | 120-132 | 133-145 |
| red birch | 131-155 | 122-140 | 130-148 |
| casuarina glauca | 127-142 | 133-151 | 140-150 |
| bayberry | 173-211 | 169-198 | 171-221 |
| fragrant fernwood | 106-122 | 109-118 | 106-120 |
| buckthorn | 135-170 | 117-123 | 125-133 |
| sea-buckthorn | 107-121 | 117-139 | 120-141 |
| oleaster | 105-120 | 120-135 | 132-150 |
| coriaria sinica | 130-141 | 137-171 | 138-161 |
| dryas octopetala | 107-115 | 105-109 | 110-118 |
| cycas szechuanensis | 105-117 | 108-153 | 117-181 |
| cycas | 108-121 | 119-147 | 120-138 |
| panzhihuaensis | | | |

7.2 In laboratory culture pots, equal area of Azolla (Salviniaceae, fern) was transplanted into a culture solution containing anabaena and lipopeptide Composition 1-7. The clean water containing anabaena was used as a control group. After cultivating for 7 days, the weight of Azolla was counted, and the data were normalized by using the data of the groups treated with clean water as standard. The results were shown in Table 3.

In the experimental field covered with hornwort, Blasia pusilla and lichen, equal area of test areas was divided. The clean water was used as a control, and lipopeptide Composition 1-7 were irrigated at 100-1000 mL per area monthly. 4 weeks later, the plants in the area were sampled and organized. The plants were weighed for their fresh weight. The data were normalized by using the data of the groups treated with clean water as standard. The results were shown in Table 3.

TABLE 3

| | Plants fresh weight % |
|---|---|
| | (The normalization processing results by using |
| | the data of clean water control groups as |
| Plants | standard) |
| Azolla | 183-461 |
| hornwort | 191-337 |
| Blasia pusilla | 144-189 |
| lichen | 201-373 |

What mentioned above are only preferred embodiments of the present disclosure, but are not intended to limit the present disclosure in any way. Although some specific embodiments of the present disclosure have been described above, those skilled in the art understand that these are merely illustrative examples. The scope of the present disclosure is defined by the appended claims. Those skilled in the art can make various changes or modifications to the technical solutions disclosed above without departing from the true scope and spirit of the disclosure, and these changes and modifications are all intended to fall within the scope of the present disclosure.

1-30. (canceled)

31. A composition for promoting root nodule growth, comprising:
(i) 0.001-100 g/L of lipopeptide; and
(ii) an adjuvant.

32. The composition according to claim 31, wherein the lipopeptide is cyclic or linear lipopeptide, preferably the cyclic lipopeptide has one of the following formulas (I) to (III): 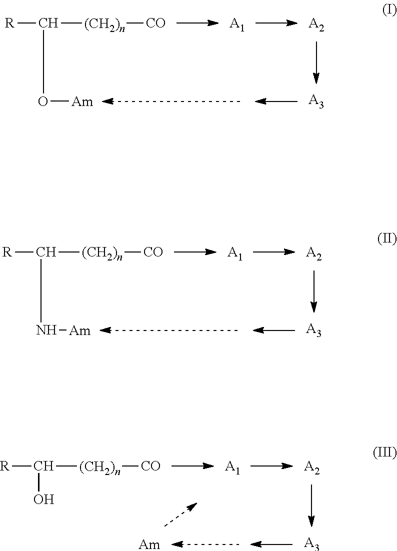
wherein R represents the carbon chain of the fatty acid chain; A₁, A₂, A₃ . . . Aₘ represent the 1^{st}, 2^{nd}, 3^{rd} . . . m^{th} amino acids on the peptide chain, respectively; the carboxyl group of the fatty acid is connected to the N-terminus of A₁, and the C-terminal carboxyl group of Aₘ is connected to the hydroxyl group or amino group of the fatty acid or other amino acids on the peptide chain to form a cyclic structure; and
the linear lipopeptide is a linear product obtained by opening the ring of cyclic lipopeptide.

33. The composition according to claim 31, wherein the lipopeptide is one or more selected from the group consisting of: surfactin family, iturin family and fengycin family, preferably the lipopeptide is one or more selected from a group consisting of: surfactin, iturin, fengycin, lichenysin, bamylocin, pumilacidin, bacillomycin, mycosubtilin, plipastatin, fusaricidin, kurstakin, paenibacterin, polymyxin, octapeptin, bacaucin and polypeptin.

34. The composition according to claim 31, wherein the lipopeptide is produced by bacteria, actinomycetes, fungi, or cyanobacteria; preferably the bacteria are wild-type bacteria or genetically engineered bacteria, and more preferably the bacteria are selected from the group consisting of: Bacillus, Pseudomonas, Streptomyces, Arthrobacter and Burkholderia; more preferably the actinomycetes are selected from the group consisting of: Streptomyces, Amycolatopsis and Microbacterium; more preferably the fungi are selected from the group consisting of: Aspergillus, Microascus, Stachylidium, Myrothecium, Sarocladium, Ophiosphaerella and Clavariopsis; more preferably the cyanobacteria are selected from the group consisting of: Okeania, Cladonia and Moorea; more preferably the lipopeptides are obtained by cultivating Bacillus or Pseudomon; and more preferably the lipopeptides are the ones obtained by cultivating Bacillus and regulating the expression of genes related to lipopeptide synthesis; and more preferably, the genes related to lipopeptide synthesis are selected from the group consisting of: lipopeptide synthesis genes, transmembrane transport protein gene ycxA, biotin carboxylase gene yngH, spore synthesis genes spoIVA B C/F, spoVA B D E, and leucine synthesis pathway genes leuABCD ilvK.

35. The composition according to claim 34, wherein the lipopeptide synthesis genes are selected from the group consisting of: surfactin synthesis gene srfA, fengycin synthesis gene fen, lichenysin synthesis gene lic, bacillomycin synthesis gene bam, mycosubtilin myc, iturin synthesis gene itu, plipastatin synthesis gene pps, fusaricidinfus, kurstakin krs, polymyxin pmx and octapeptin oct.

36. The composition according to claim 31, further comprising 0.01-99.99 wt % of one or more components selected from the group consisting of: polysaccharides, oligosaccharides, dextrins, amino acids, oligopeptides, proteins, lipoids, fatty acids or the derivatives thereof, inorganic salts, dispersants, wetting agents, polymers, and warning colorants.

37. The composition according to claim 31, wherein the composition is a culture solution obtained by cultivating bacteria capable of producing lipopeptides.

38. The composition according to claim 31, wherein the root nodule is produced by bacteria, actinomycetes, fungi, or cyanobacteria that are symbiotic with plants; preferably the bacteria are selected from α-, β-, or γ-Proteobacteria, and more preferably selected from the group consisting of: Rhizobium, Sinorhizobium, Ensifer, Shinella, Neorhizobium, Pararhizobium, Mesorhizobium, Bradyrhizobium, Phyllobacterium, Methylobacterium, Microvirga, Ocrhobactrum, Azorhizobium, Devosia, Burkholderia, Cupriavidu and Pseudomonas; preferably the actinomycetes are Frankia; preferably the fungi are Ascomycetes and Basidiomycetes; preferably the cyanobacteria are Nostoc and Anabaena.

39. The composition according to claim 38, wherein the root nodule is produced by bacteria that are symbiotic with plants; and the plants are leguminous or cannabis family of plants, preferably selected from the group consisting of: soybeans, broad beans, peas, mung beans, adzuki beans, cowpeas, kidney beans, lentils, winged beans, black beans, chickpeas, peanuts, purple vetch, alfalfa, Chinese milk vetch, acacia, Dalbergia hupeana Hance, Chinese honey locust, Erythrophleum fordii Oliv., Sophora japonica, Indigofera pseudotinctoria, Indigofera tinctoria L, sappan wood, arabic tree gum, Astragalus membranaceus, copal gum tree, Crotalaria juncea L., kudzu vine, Trema tomentosa, and Parasponia.

40. The composition according to claim 48, the root nodule is produced by Frankia, cyanobacteria or fungi that are symbiotic with plants, and the plants are selected from the group consisting of: Betulaceae, Casuarinaceae, Myricaceae, Rhamnaceae, Elaeagnaceae, Datiscaceae, Coriariaceae, Rosaceae, Bryophytes, ferns and cycades, Nostoc, Coccidium, Trentepohlia, Scytonema and Scytonema.

41. A method for promoting root nodule growth by using the composition according to claim 31, comprising:
(1) directly applying the composition to the culture of the plants that are symbiotic with bacteria, actinomycetes, fungi, or cyanobacteria which produce the root nodule;
(2) cultivating and harvesting the plants;
(3) optionally measuring the parameters of the root nodule, and comparing them with those of the control group to which no composition is applied, preferably, the parameters are selected from the group consisting of: the number of root nodules and root nodule weight of each plant.

42. The method according to claim 41, wherein the promotion of root nodule growth is characterized by one or more features selected from the group consisting of:
(1) the number of root nodules per plant of the plants cultivated by applying the composition is at least 1.1 times, at least 1.2 times, at least 1.3 times, at least 1.4 times, at least 1.5 times, at least 1.6 times, at least 1.7 times, at least 1.8 times, at least 1.9 times, at least 2 times, preferably at least 3 times, more preferably at least 4 times, more preferably at least 5 times, more preferably at least 6 times, more preferably at least 7 times that of the control group; and/or
(2) the root nodule weight per plant of the plants cultivated by applying the composition is at least 1.1 times, at least 1.2 times, at least 1.3 times, at least 1.4 times, at least 1.5 times, at least 1.6 times, at least 1.7 times, at least 1.8 times, at least 1.9 times, at least 2 times, preferably at least 3 times, more preferably at least 4 times, more preferably at least 5 times, more preferably at least 6 times, more preferably at least 7 times that of the control group.

43. The method according to claim 41, which meanwhile promotes the growth of the plants that are symbiotic with bacteria, actinomycetes, fungi, or cyanobacteria which produce the root nodule, preferably the promotion of the growth of the plants is characterized by the plant height of the aboveground part, the fresh weight of the aboveground part, the fresh weight of the underground part, and the number or weight of plant products; more preferably is characterized by one or more features selected from the group consisting of:
(1) the plant height of the aboveground part of the plants cultivated by applying the composition increases 10% or more, preferably 15% or more, more preferably 20% or more, more preferably 25% or more, more preferably 30% or more, more preferably 35% or more, more preferably 36% or more, as compared to the control group;
(2) the fresh weight of the more ground part of the plants cultivated by applying the composition increases 15% or more, preferably 20% or more, more preferably 25% or more, more preferably 30% or more, more preferably 35% or more, more preferably 40% or more more preferably 45% or more, more preferably 47% or more, as compared to the control group;
(3) the fresh weight of the underground part of the plants cultivated by applying the composition increases 5% or more, preferably 8% or more, more preferably 10% or more, more preferably 12% or more, as compared to the control group; and/or
(4) the number or weight of plant products cultivated by applying the composition is at least 1.1 times, preferably at least 1.3 times, more preferably at least 1.6 times, more preferably at least 1.9 times, more preferably at least 2.3 times, more preferably at least 3.0 times that of the control group; and preferably the plant products are selected from the group consisting of: fruit pods, pegs and seeds of the plant.

44. The method according to claim 41, wherein the application is selected from the group consisting of: seed coating, seed soaking treatment, bulb treatment, root irrigation treatment, drip irrigation treatment, and foliar spraying application.

45. A method for promoting root nodule growth by using lipopeptide, comprising:
(1) directly applying the lipopeptide to the culture of the plants that are symbiotic with bacteria, actinomycetes, fungi, or cyanobacteria which produce the root nodule;
(2) cultivating and harvesting the plants;
(3) optionally measuring the parameters of the root nodule, and comparing them with those of the control group to which no lipopeptide is applied, preferably, the parameters are selected from the group consisting of: the number of root nodules and root nodule weight of each plant.

46. The method according to claim 45, which meanwhile promotes the growth of the plants that are symbiotic with bacteria, actinomycetes, fungi, or cyanobacteria which produce the root nodule, preferably the promotion of the growth of the plants is characterized by the plant height of the aboveground part, the fresh weight of the aboveground part, the fresh weight of the underground part, and the number or weight of plant products; more preferably is characterized by one or more features selected from the group consisting of:
(1) the plant height of the aboveground part of the plants cultivated by applying the lipopeptide increases 10% or more, preferably 15% or more, more preferably 20% or more, more preferably 25% or more, more preferably 30% or more, more preferably 35% or more more preferably 36% or more, as compared to the control group;
(2) the fresh weight of the more ground part of the plants cultivated by applying the lipopeptide increases 15% or more, preferably 20% or more, more preferably 25% or more, more preferably 30% or more, more preferably 35% or more, more preferably 40% or more, more preferably 45% or more, more preferably 47% or more, as compared to the control group;
(3) the fresh weight of the underground part of the plants cultivated by applying the lipopeptide increases 5% or more, preferably 8% or more, more preferably 10% or more, more preferably 12% or more, as compared to the control group; and/or
(4) the number or weight of plant products cultivated by applying the lipopeptide is at least 1.1 times, preferably at least 1.3 times, more preferably at least 1.6 times, more preferably at least 1.9 times, more preferably at least 2.3 times, more preferably at least 3.0 times that of the control group; and preferably the plant products are selected from the group consisting of: fruit pods, pegs and seeds of the plant;
preferably, wherein the application is selected from the group consisting of: seed coating, seed soaking treatment, bulb treatment, root irrigation treatment, drip irrigation treatment, and foliar spraying application.

47. The method according to claim 45, wherein the lipopeptide is one or more selected from the group consisting of: surfactin, iturin, fengycin, lichenysin, bamylocin, pumilacidin, bacillomycin, mycosubtilin, plipastatin, fusaricidin, kurstakin, paenibacterin, polymyxin, octapeptin, bacaucin and polypeptin.

48. The method according to claim 45, wherein the lipopeptide is produced by bacteria, actinomycetes, fungi, or cyanobacteria; preferably the bacteria are wild-type bacteria or genetically engineered bacteria, and more preferably the bacteria are selected from the group consisting of: Bacillus, Pseudomonas, Streptomyces, Arthrobacter and Burkholderia; more preferably the actinomycetes are selected from the group consisting of: Streptomyces, Amycolatopsis and Microbacterium; and more preferably the fungi are selected from the group consisting of: Aspergillus, Microascus, Stachylidium, Myrothecium, Sarocladium, Ophiosphaerella and Clavariopsis; and more preferably the cyanobacteria are selected from the group consisting of: Okeania, Cladonia and Moorea; more preferably the lipopeptides are obtained by cultivating Bacillus or Pseudomonas; and more preferably the lipopeptides are the ones obtained by cultivating Bacillus and regulating the expression of genes related to lipopeptide synthesis; and more preferably, the genes related to lipopeptide synthesis are selected from the group consisting of: lipopeptide synthesis genes, transmembrane transport protein gene ycxA, biotin carboxylase gene yngH, spore synthesis genes spoIVA B C/F, spoVA B D E, and leucine synthesis pathway genes leuABCD ilvK;
alternatively, wherein the lipopeptide synthesis genes are selected from the group consisting of: surfactin synthesis gene srfA, fengycin synthesis gene fen, lichenysin synthesis gene lic, bacillomycin synthesis gene bam, mycosubtilin myc, iturin synthesis gene itu, plipastatin synthesis gene pps, fusaricidinfus, kurstakin krs, polymyxin pmx and octapeptin oct.

49. The method according to claim 45, wherein the root nodule is produced by bacteria, actinomycetes, fungi, or cyanobacteria that are symbiotic with plants; and wherein preferably the bacteria are selected from α-, β-, or γ-Proteobacteria, and more preferably are selected from the group consisting of: Rhizobium, Sinorhizobium, Ensifer, Shinella, Neorhizobium, Pararhizobium, Mesorhizobium, Bradyrhizobium, Phyllobacterium, Methylobacterium, Microvirga, Ocrhobactrum, Azorhizobium, Devosia, Burkholderia, Cupriavidu and Pseudomonas; preferably the actinomycetes are Frankia; preferably the fungi are Ascomycetes and Basidiomycetes; preferably the cyanobacteria are Nostoc and Anabaena;
alternatively, wherein the root nodule is produced by bacteria that are symbiotic with plants; and the plants are leguminous or cannabis plants, preferably selected from the group consisting of: soybeans, broad beans, peas, mung beans, adzuki beans, cowpeas, kidney beans, lentils, winged beans, black beans, chickpeas, peanuts, purple vetch, alfalfa, Chinese milk vetch, acacia, Dalbergia hupeana Hance, Chinese honey locust, Erythrophleum fordii Oliv., Sophora japonica, Indigofera pseudotinctoria, Indigofera tinctoria L, sappan wood, arabic gum tree, Astragalus membranaceus, copal gum tree, Crotalaria juncea L., kudzu vine, Trema tomentosa, and Parasponia;
alternatively, wherein the root nodule is produced by Frankia, cyanobacteria or fungi that are symbiotic with plants, and the plants are selected from the group consisting of: Betulaceae, Casuarinaceae, Myricaceae, Rhamnaceae, Elaeagnaceae, Datiscaceae, Coriariaceae, Rosaceae, Bryophytes, ferns and cycades, Nostoc, Coccidium, Trentepohlia, Scytonema and Scytonema.

50. A method for promoting root nodule growth and meanwhile promoting the growth of the plants that are symbiotic with bacteria, actinomycetes, fungi, or cyanobacteria which produce the root nodule, by using lipopeptide, comprising:
(1) directly applying the lipopeptide to the culture of the plants that are symbiotic with bacteria, actinomycetes, fungi, or cyanobacteria which produce the root nodule;
(2) cultivating and harvesting the plants;
(3) optionally measuring the parameters of the root nodule, and comparing them with those of the control group to which no lipopeptide is applied, preferably, the parameters are selected from the group consisting of: the number of root nodules and root nodule weight of each plant.
